(19) United States
(12) Patent Application Publication
(10) Pub. No.: US 2020/0261063 A1
(43) Pub. Date: Aug. 20, 2020

(54) ULTRASONIC PROBE AND ULTRASONIC DIAGNOSTIC DEVICE

(71) Applicant: Hitachi, Ltd., Tokyo (JP)

(72) Inventor: Koichi ISHIDA, Tokyo (JP)

(73) Assignee: Hitachi, Ltd., Tokyo (JP)

(21) Appl. No.: 16/737,465

(22) Filed: Jan. 8, 2020

(30) Foreign Application Priority Data
Feb. 14, 2019 (JP) 2019-024434

(51) Int. Cl. A61B 8/00; A61B 8/12

(52) U.S. Cl. CPC A61B 8/546; A61B 8/12

(57) ABSTRACT

A connector has a bottom wall located at a lower portion in an attached state in which the connector is attached to an ultrasonic diagnostic device body. The bottom wall has a ventilation portion having a plurality of openings and a non-ventilation portion adjacent to the ventilation portion. The bottom wall has an inner step that functions as a weir structure for restriction of movement of liquid from an inner face of the non-ventilation portion to an inner face of the ventilation portion in the attached state. An outer face of the bottom wall is provided with an outer step corresponding to the inner step.

### BACKGROUND OF THE INVENTION

### 1. Field of the Invention

The present invention relates to an ultrasonic probe and an ultrasonic diagnostic device, and particularly relates to the structure of an ultrasonic probe provided with a coolant circulation system.

### 2. Description of the Related Art

An ultrasonic diagnostic device is a medical device that forms an ultrasonic image, on the basis of a received signal acquired by ultrasonic transmission and reception to a living body. Such an ultrasonic diagnostic device typically includes a device body and an ultrasonic probe. Recently, an ultrasonic probe (3D probe) capable of performing two-dimensional electronic scanning with an ultrasonic beam, has been commercially practical. Generally, such an ultrasonic probe has a probe head to be grasped by an examiner (hereinafter, simply referred to as a “head”) in which an electronic circuit for generation of a transmission signal and processing of a received signal is provided. For example, the electronic circuit includes a plurality of integrated circuits. The electronic circuit provided in the head acts as a heat source. In addition, an ultrasonic transducer provided in the head acts as a heat source.

In order to inhibit such a head (particularly, a transmission and reception face to be in contact with a living body) from rising in temperature, an ultrasonic probe provided with a coolant circulation system (coolant-circulation ultrasonic probe) has been proposed (refer to JP 2008-149135 A). The coolant circulation system circulates a coolant between a head and a probe connector (hereinafter, simply referred to as a “connector”) to carry heat generated in the head to the connector, so that the head is inhibited from rising in temperature.

### SUMMARY OF THE INVENTION

In the coolant-circulation ultrasonic probe, coolant equipment including a heat exchanger and a pump is provided in the connector. The coolant equipment is connected with two supplying tubes extending from the head. A plurality of components included in the coolant equipment is mutually connected through laid pipes, such as tubes. In some cases, a connection is made between laid pipes in the probe connector. In this manner, usually, a plurality of laid-pipe connections, namely, a plurality of joints is present in the connector.

For each individual joint, a countermeasure against leakage of the coolant at the joint has been taken. Even if the coolant leaks at any joint in the connector, from the viewpoint of safety, desirably, the coolant does not leak out of the connector. Note that, JP 2008-149135 A discloses a coolant-circulation ultrasonic probe, but no specific structure has been described for the housing of a connector in JP 2008-149135 A.

An object of the present invention is to provide a coolant-circulation ultrasonic probe enabling a coolant leaking in a connector, to be maximally inhibited from leaking out of the connector. Another object of the present invention is to provide an ultrasonic diagnostic device including a coolant-circulation ultrasonic probe, enabling maintenance or improvement of safety.

An ultrasonic probe according to an embodiment of the present invention, includes:

a head configured to perform ultrasonic transmission and reception; and

a connector connected to the head through a cable, the connector being to be detachably attached to an ultrasonic diagnostic device body, the connector including coolant equipment in which a coolant for head cooling flows and a housing housing the coolant equipment, in which

the housing has a bottom wall located at a lower portion in an attached state in which the connector is attached to the ultrasonic diagnostic device body,

the bottom wall has a ventilation portion having a plurality of openings and a non-ventilation portion adjacent to the ventilation portion, and

the bottom wall has a weir structure for restriction of movement of liquid from an inner face of the non-ventilation portion to an inner face of the ventilation portion in the attached state.

An ultrasonic diagnostic device according to an embodiment of the present invention, includes:

an ultrasonic diagnostic device body; and

an ultrasonic probe including: a head that performs ultrasonic transmission and reception; and a connector to be detachably attached to the ultrasonic diagnostic device body, in which

the connector includes coolant equipment in which a coolant flows and a housing housing the coolant equipment,

the housing has a bottom wall located at a lower portion in an attached state in which the connector is attached to the ultrasonic diagnostic device body,

the bottom wall has a ventilation portion having a plurality of openings and a non-ventilation portion adjacent to the ventilation portion, and

the bottom wall has a weir structure for restriction of movement of liquid from an inner face of the non-ventilation portion to an inner face of the ventilation portion in the attached state.

According to the embodiments of the present invention, the coolant-circulation ultrasonic probe enables, even if the coolant leaks in the connector, prevention of leakage of the coolant outside the connector or decrease of the possibility of the leakage. The ultrasonic diagnostic device including the coolant-circulation ultrasonic probe enables maintenance or improvement of safety.

### BRIEF DESCRIPTION OF THE DRAWINGS

FIG. 1 is a block diagram of an ultrasonic diagnostic device according to an embodiment;

FIG. 2 is a first perspective view of a connector according to a first embodiment;

FIG. 3 is a second perspective view of the connector according to the first embodiment;

FIG. 4 is a plan view of the inside of the connector according to the first embodiment;

FIG. 5 is a perspective view of a lower case according to the first embodiment;

FIG. 6 is a perspective view of coolant equipment according to the first embodiment;

FIG. 7 is a schematic xz sectional view of the lower case according to the first embodiment;

FIG. 8 is a yz sectional view of the lower case according to the first embodiment;

FIG. 9 is a view of a modification of a weir structure;

FIG. 10 is a view for describing exemplary arrangement of absorbing members and pump control;

FIG. 11 is a first perspective view of a connector according to a second embodiment;

FIG. 12 is a second perspective view of the connector according to the second embodiment;

FIG. 13 is a perspective view of a first case according to the second embodiment; and

FIG. 14 is a schematic xz sectional view of the first case according to the second embodiment.

### DESCRIPTION OF THE PREFERRED EMBODIMENTS

Embodiments will be described below on the basis of the drawings.

### (1) Outline of Embodiments

An ultrasonic probe according to an embodiment includes: a head that performs ultrasonic transmission and reception; and a connector connected to the head through a cable, the connector being to be detachably attached to an ultrasonic diagnostic device body. The connector includes: coolant equipment in which a coolant for head cooling flows; and a housing housing the coolant equipment. In the attached state in which the connector is attached to the ultrasonic diagnostic device body, the housing has a bottom wall located at the lower portion thereof. The bottom wall has a ventilation portion having a plurality of openings and a non-ventilation portion adjacent to the ventilation portion. Furthermore, the bottom wall has a weir structure for restriction of movement of liquid from the inner face of the non-ventilation portion to the inner face of the ventilation portion in the attached state.

According to the configuration above, the weir structure is provided between the non-ventilation portion and the ventilation portion. Thus, in a case where the coolant drops onto or flows to the inner face of the non-ventilation portion, movement of the coolant from the inner face of the non-ventilation portion to the inner face of the ventilation portion is restricted. This arrangement enables prevention of leakage of the coolant outside the housing or decrease of the leakage. The weir structure has an effect of damming the coolant such that the coolant does not move naturally. For example, the weir structure may be formed of a step, a bulkhead, or a partition. The inner face of the ventilation portion is a ventilation area to be described later, and the inner face of the non-ventilation portion is a non-ventilation area to be described later.

Usually, coolant circulation is performed at ultrasonic diagnosis. At the coolant circulation, the pressure in a coolant channel rises. Particularly, the pressure rises on the output side of a pump or in the vicinity thereof. Increase of the pressure causes increase of the risk of leakage of the coolant. Meanwhile, prevention or decrease of outward leakage of the coolant is desirable at ultrasonic diagnosis. This is because a subject and an examiner are present near the ultrasonic diagnostic device body or the ultrasonic probe.

Furthermore, this is because, in some cases, an electronic apparatus is present near the ultrasonic diagnostic device body or the ultrasonic probe. From the viewpoint as above, on condition that the ultrasonic probe is attached, the configuration above enables prevention or decrease of outward leakage of the coolant in the attached state.

According to the embodiment, the inner face of the ventilation portion is located higher than the inner face of the non-ventilation portion in the attached state. In that case, the weir structure includes an inner step between the inner face of the ventilation portion and the inner face of the non-ventilation portion. According to this configuration, the inner step dams the coolant. In a case where the inner face itself of the ventilation portion is high in location, even if the coolant enters on the ventilation portion side over the weir structure, the coolant is guided to the non-ventilation portion naturally. Therefore, even if the coolant leaks outside through any of the openings, the leakage of the coolant is little.

According to the embodiment, the outer face of the ventilation portion is located higher than the outer face of the non-ventilation portion in the attached state. Thus, an outer step is provided between the outer face of the ventilation portion and the outer face of the non-ventilation portion. At grasping of the connector, if one or a plurality of fingers catches the outer step, the connector can be grasped securely. Therefore, at attachment of the connector and at detachment of the connector, operability or workability improves.

According to the embodiment, the coolant equipment includes a plurality of joints. In the attached state, the plurality of joints is located apart from the space right above the inner face of the ventilation portion. According to this configuration, even if the coolant bleeds from any of the joints (laid-pipe connections), the coolant can be prevented from dropping onto the ventilation portion. In a case where the coolant drops onto the non-ventilation portion, movement of the coolant from the inner face of the non-ventilation portion to the inner face of the ventilation portion is restricted by the weir structure above.

The ultrasonic probe according to the embodiment includes a substrate array of a plurality of substrates. In the attached state, the substrate array is deviated upward in location in the housing. The substrate array is spaced apart from the bottom wall, so that the electrical soundness of the substrate array is maintained easily. In the height direction, in a case where the center of the substrate array is higher in height than the center of the housing, it can be said that the substrate array is biased upward in the housing.

In the attached state, a certain gap secured between the inner face of the bottom wall and the lowermost face of the substrate array enables prevention of the coolant on the bottom wall from influencing the substrate array. Meanwhile, in a case where the connector is detached from the ultrasonic diagnostic device body, the attitude of the connector is variable. In consideration of such a circumstance, preferably, the substrate array is spaced apart from the inner face of the bottom wall as much as possible or to a certain extent. In order to protect the substrate array further, desirably, the substrate array is covered with a case. As described below, desirably, an absorbing member that absorbs the coolant leaking is provided in the connector.

The ultrasonic probe according to the embodiment includes an absorbing member that absorbs the coolant leaking, provided in the housing. According to this configuration, the coolant leaking can be prevented from flowing freely in the connector, so that the coolant is stored in a specific place in the housing. The absorbing member is generally a porous member, and thus the coolant after absorption can be expected to evaporate.

According to the embodiment, the ventilation portion is intended for intake. According to this configuration, outer air is sucked inside the housing through each individual opening. Thus, wind pressure is applied to the coolant having entered each individual opening so that the coolant is prevented from moving forward. Desirably, a ventilation portion for discharge is provided at a high location apart by a certain distance from the inner face of the bottom wall so that no coolant flows unexpectedly through the ventilation portion for discharge in the attached state.

An ultrasonic diagnostic device according to an embodiment includes an ultrasonic diagnostic device body and an ultrasonic probe. The ultrasonic probe includes: a head that performs ultrasonic transmission and reception; and a connector to be detachably attached to the ultrasonic diagnostic device body. The connector includes: coolant equipment in which a coolant flows; and a housing housing the coolant equipment. In the attached state in which the connector is attached to the ultrasonic diagnostic device body, the housing has a bottom wall located at the lower portion thereof. The bottom wall has a ventilation portion having a plurality of openings and a non-ventilation portion adjacent to the ventilation portion. The bottom wall has a weir structure for restriction of movement of liquid from the inner face of the non-ventilation portion to the inner face of the ventilation portion in the attached state.

According to the embodiment, the ultrasonic probe includes a detector that detects leakage of the coolant in the housing, and the ultrasonic probe or the ultrasonic diagnostic device body includes a control unit that controls the operation of the coolant equipment, on the basis of a detected signal output from the detector. According to this configuration, in a case where the coolant leaks, a measure such as stopping of a pump is carried out. The control unit may generate an alarm.

### (2) Details of Embodiments

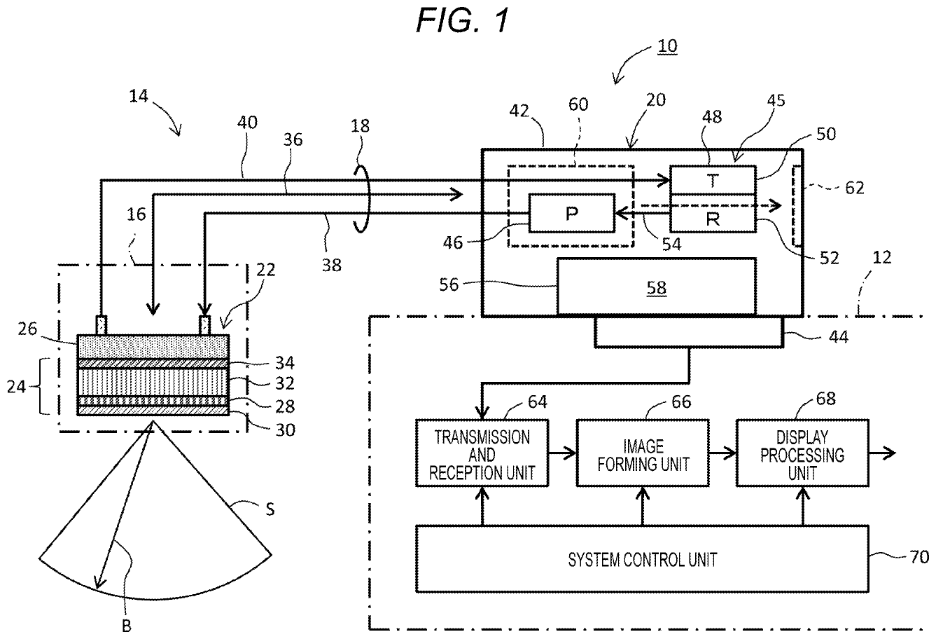

FIG. 1 illustrates an ultrasonic diagnostic device according to an embodiment. The ultrasonic diagnostic device is a medical device that is provided at a medical institution, such as a hospital, and forms and displays an ultrasonic image, on the basis of a received signal acquired by ultrasonic transmission and reception to a living body. The ultrasonic diagnostic device 10 illustrated that is a cart-type ultrasonic diagnostic device, includes a device body (ultrasonic diagnostic device body) 12 and an ultrasonic probe 14.

First, the ultrasonic probe 14 will be described. The ultrasonic probe 14 is a 3D probe provided with a coolant circulation system. Specifically, the ultrasonic probe 14 includes a head (probe head) 16, a cable (probe cable) 18, and a connector (probe connector) 20. The head 16 to be held by a user (e.g., a doctor or an examination technician) performs ultrasonic transmission and reception. The transmission and reception face of the head 16 is made in contact with the surface of a living body. Ultrasonic diagnosis is carried out in that state.

The head 16 includes an assembly 22. The assembly 22 includes a stack 24 and a cooling jacket 26. The cooling jacket 26 is provided on the back side of the stack 24 (no-living-body side). The stack 24 includes a two-dimensional vibrating-element array 28. The two-dimensional vibrating-element array 28 is formed of hundreds, thousands, tens of thousands of vibrating elements, or more. For example, the vibrating elements are two-dimensionally arrayed in a planar shape. For example, a matching layer and a protective layer are provided on the living-body side of the two-dimensional vibrating-element array 28 (refer to reference numeral 30). The surface of the protective layer serves as the transmission and reception face to be in contact with a living body. For example, a backing 32 is provided on the back side of the two-dimensional vibrating-element array 28. A lead array is embedded in the backing 32. The lead array is formed of a plurality of leads for electrical connection between the two-dimensional vibrating-element array 28 and an electronic circuit 34 to be described below.

The electronic circuit 34 is provided on the back side of the backing 32. In the illustrated exemplary configuration, the electronic circuit 34 includes a plurality of integrated circuits (ICs). The electronic circuit 34 has a function of generating a plurality of transmission signals and a function of processing a plurality of received signals. Specifically, the electronic circuit 34 has a function of performing sub-beamforming for channel reduction. Along with the operation of the electronic circuit 34, a relatively large amount of heat is generated. Heat is generated at the two-dimensional vibrating-element array 28.

The coolant circulation system serves as means for carrying the heat to the connector 20 side. In the illustrated exemplary configuration, the coolant circulation system includes the cooling jacket 26 in the head 16, two tubes 38 and 40 in the cable 18, and coolant equipment 45 in the connector 20. For example, the coolant is a liquid containing water or oil as a main component.

The two tubes 38 and 40 are connected to the cooling jacket 26 that is a heat exchanger. Specifically, the coolant having been cooled is fed from the tube 38 to the cooling jacket 26, so that endothermic effect is achieved at the cooling jacket 26. The coolant having risen in temperature is fed from the cooling jacket 26 to the tube 40.

The two-dimensional vibrating-element array 28 forms an ultrasonic beam B. Electronic scanning is performed in a first direction with the ultrasonic beam B, resulting in formation of a beam scanning plane S that is a two-dimensional data capturing space. Electronic scanning is performed in a second direction with the beam scanning plane S, resulting in formation of a three-dimensional data capturing space. Examples of an electronic scanning system that have been known, include an electronic sector scanning system and an electronic linear scanning system.

Note that a plurality of vibrating elements may be arrayed along a cylindrical face. A one-dimensional vibrating-element array may be used. Instead of the ultrasonic probe 14 that is a body-surface contact type, an ultrasonic probe that is a body-cavity insertion type may be used.

The head 16 and the connector 20 are connected through the cable 18. The cable 18 includes a signal-line group 36 connected to the electronic circuit 34. The cable 18 includes the two tubes 38 and 40 as above.

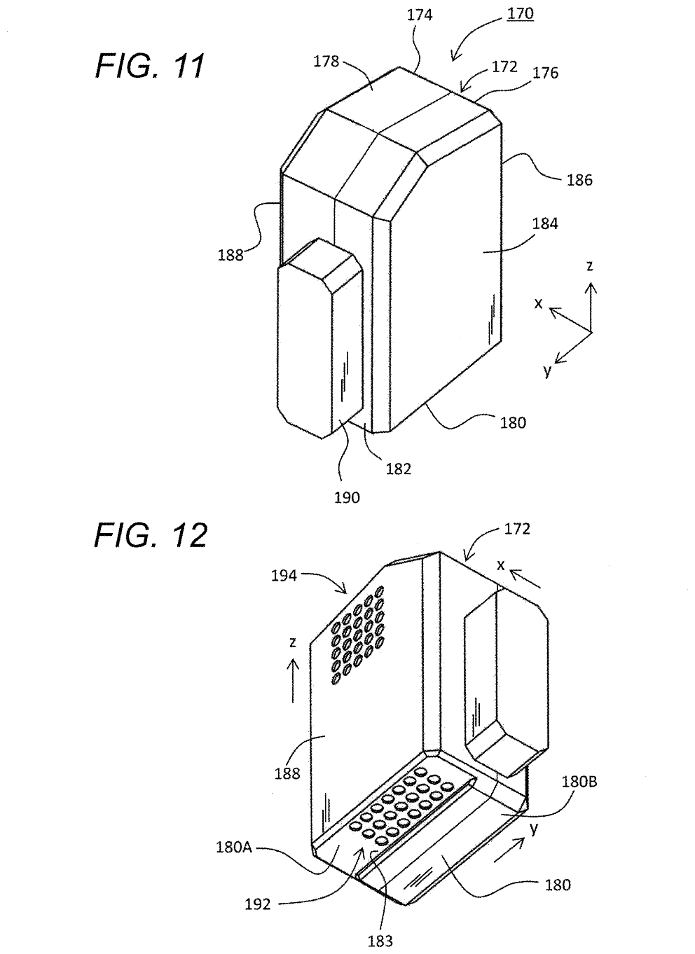

The connector 20 is detachably attached to the device body 12. The connector 20 is substantially a rectangular parallelepiped. A horizontal attitude or a vertical attitude is considered as the attitude in the attached state. The connector 20 according to a first embodiment illustrated in FIGS. 1 to 10 is attached to the device body 12 in the horizontal attitude. Meanwhile, a connector 170 according to a second embodiment illustrated in FIGS. 11 to 14, is attached to the device body in the vertical attitude.

In FIG. 1, the connector 20 includes a housing 42. For example, the coolant equipment 45 and a substrate array 58 are housed in the housing 42. The housing 42 is provided with a terminal portion 44 including a terminal group. The terminal portion 44 is inserted in a receptacle provided on the device body 12 side. This arrangement causes the connector 20 to be physically and electrically connected to the device body 12. The receptacle extends horizontally. Note that, usually, a plurality of receptacles is provided at the device body 12.

In the illustrated exemplary configuration, the coolant equipment 45 includes a pump 46, a composite component 48, and a tube 54. The composite component 48 includes a tank 50 and a radiator 52. The tank 50 stores a certain amount of coolant. The radiator 52 that is a heat exchanger, includes a fin array for heat dissipation. The tube 40 is connected to the tank 50. The coolant having risen in temperature fed through the tube 40, flows in the radiator 52 through the tank 50. The coolant drops in temperature due to heat exchange by the radiator 52. The coolant having been cooled is fed to the pump 46 through the tube 54. The pump 46 is connected with the tube 38. The pump 46 generates coolant carrying power.

In the illustrated exemplary configuration, the pump 46 is provided on the downstream side of the radiator 52. However, the pump 46 may be provided on the upstream side of the radiator 52. In the illustrated exemplary configuration, the tank 50 and the radiator 52 are integrally formed. However, the tank 50 and the radiator 52 may be separately provided and may be connected through a laid pipe. According to the illustrated exemplary configuration, because the tank 50 and the radiator 52 are integrally formed, the number of components and the number of joints can be reduced, and additionally miniaturization is achieved.

An intake portion 60 and a discharge portion 62 are provided at the connector 20. The intake portion 60 includes a ventilation portion having a plurality of openings provided at the housing and a filter provided inside the ventilation portion. Similarly, the discharge portion 62 includes a ventilation portion having a plurality of openings provided at the housing and a filter provided inside the ventilation portion.

For example, a blower fan and a duct member, not illustrated in FIG. 1, are provided in the housing 42. Due to the effect of the blower fan, outer air is taken in the housing 42 through the intake portion 60. Then, the air is guided to the discharge portion 62 through the inside of the duct member. The fin array of the radiator is provided in the duct member, and the fin array is cooled by the air flowing in the duct member. That is heat exchange is performed between the fin array and the air. According to the first embodiment, the intake portion 60 is provided at a bottom wall as part of the housing 42. The intake portion 60 and the discharge portion 62 each may be regarded as a constituent element of the coolant circulation system. An inner step that functions as a weir structure or a weir means, is provided at the inner face of the bottom wall. This arrangement will be described later.

A box-shaped inner case 56 is provided in the housing 42. The substrate array 58 is housed in the inner case 56. The substrate array 58 is formed of a plurality of substrates. Each substrate is equipped with an electronic circuit. Examples of the electronic circuit include an electronic circuit for control of the operation of the pump 46, an electronic circuit for processing of a received signal, and an electronic circuit for control. An absorbing member is provided in the housing 42. However, the illustration thereof is omitted in FIG. 1.

Next, the device body 12 will be described. A transmission and reception unit 64 includes a transmission unit and a reception unit. The transmission unit functions as a transmission main beamformer. The transmission unit may be provided in the electronic circuit 34. The reception unit functions as a reception main beamformer. Specifically, the reception unit applies delay and sum to a plurality of received signals, to generate beam data. For example, one piece of volume data includes a plurality of pieces of frame data. One piece of frame data includes a plurality of pieces of beam data. Each individual piece of frame data corresponds to the beam scanning plane S, and each individual piece of beam data corresponds to the ultrasonic beam B.

An image forming unit 66 has a function of forming a tomographic image on the basis of each individual piece of frame data and a function of forming a three-dimensional image on the basis of the volume data. The three-dimensional image is a planar image in which tissue is expressed in three dimensions. The three-dimensional image is generated by, for example, volume rendering or surface rendering. Data of an ultrasonic image formed by the image forming unit 66 is sent to a display not illustrated through a display processing unit 68. The ultrasonic image is displayed on the display screen of the display.

A system control unit 70 that controls the operation of each constituent included in the ultrasonic diagnostic device 10, includes a CPU and a program. The system control unit 70 may include a plurality of processors that executes the program. The control items of the system control unit 70 include transmission and reception control and coolant circulation control. The coolant circulation control may be performed in the ultrasonic probe 14.

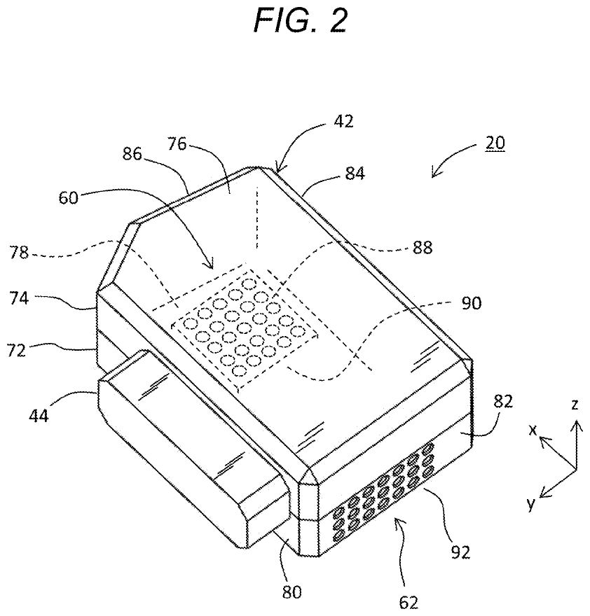

FIG. 2 illustrates the connector 20 having a horizontal attitude. The connector 20 includes the housing 42 that is box-shaped and the terminal portion 44. An x direction is the left and right direction as a first horizontal direction. A y direction is the depth direction as a second horizontal direction. A z direction is the vertical direction (perpendicular direction). The connector 20 that is tabular, expands in the x direction and the y direction. With the horizontal attitude retained, the connector 20 is attached to the device body. Alternatively, the connector 20 having been attached to the device body is in the horizontal attitude. According to the first embodiment, the horizontal attitude is the attitude in the attached state.

The housing 42 has an upper wall 76 and a lower wall (hereinafter, referred to as the bottom wall) 78, and also has four side walls 80, 82, 84, and 86. The housing 42 is provided with the terminal portion 44 protruding from the side wall 80 in the y direction. Specifically, the housing 42 is formed of a lower case 72 and an upper case 74. For example, the lower case 72 and the upper case 74 each are made of resin. Note that the inner face of the lower case 72 and the inner face of the upper case 74 each are provided with a metal frame or a metal sheet. However, the illustrations thereof are omitted in each figure.

An opening group 88 of the plurality of openings is formed at the bottom wall 78. Each individual opening that is a vertical through hole, is intended for communication between the inside and the outside of the housing 42. The opening group 88 functions as the ventilation portion. The ventilation portion is a constituent element of the intake portion 60. Each individual opening illustrated is a circular opening, but may be rectangular or different in shape.

The inner face of the bottom wall 78 is broadly divided into a ventilation area as the inner face of the ventilation portion and a non-ventilation area as the inner face of a non-ventilation portion. The ventilation area has the opening group 88 formed therein. In the attached state, namely, in a case where the connector is in the horizontal attitude, the ventilation area is higher in height than the non-ventilation area. The inner step 90 that functions as the weir structure is present between the ventilation area and the non-ventilation area. In the illustrated example, the inner step 90 is rectangular.

An opening group 92 having the plurality of openings is formed at the side wall 82. Each individual opening that is a horizontal through hole, is intended for communication between the inside and the outside of the housing 42. The opening group 92 functions as the ventilation portion. The ventilation portion is a constituent element of the discharge portion 62. Each individual opening illustrated is a circular opening, but may be rectangular or different in shape. As described later, the opening group 92 is provided at a high location apart by a certain distance from the inner face of the bottom wall 78.

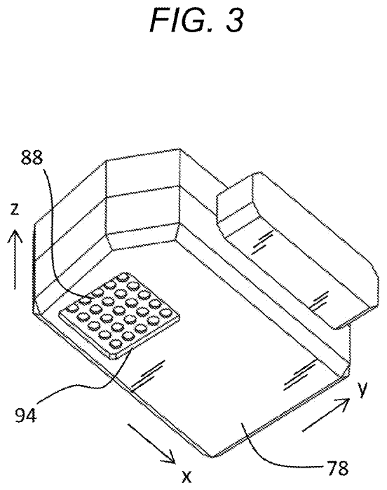

FIG. 3 illustrates the connector viewed obliquely from below. The opening group 88 included in the ventilation portion appears at the outer face of the bottom wall 78. The portion is recessed to the periphery thereof, and thus an outer step 94 is present. The outer step 94 corresponds to the inner step above, and thus is rectangular in the illustrated example.

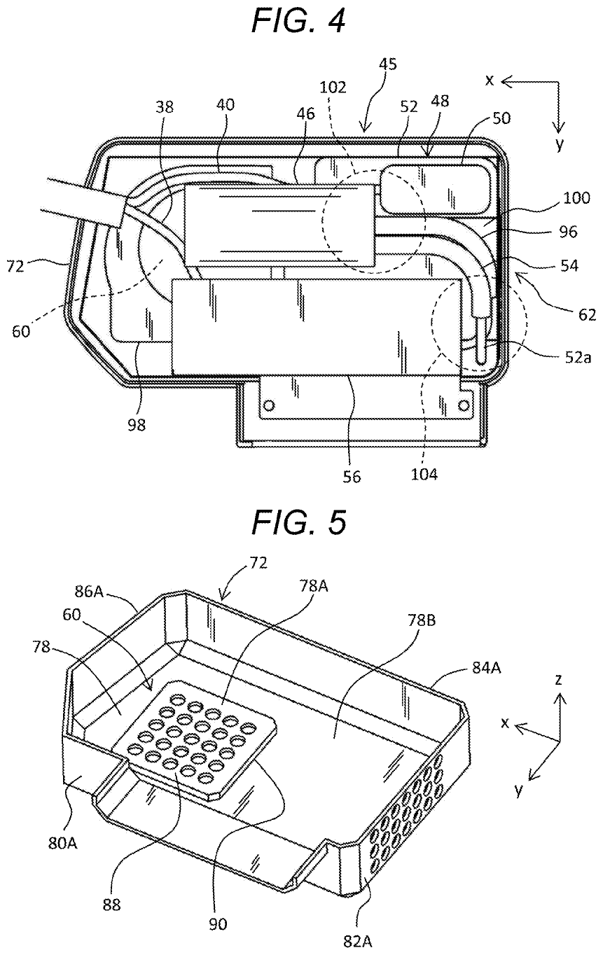

FIG. 4 illustrates the inside of the housing. In FIG. 4, because the upper case is detached, the lower case 72 appears. The coolant equipment 45 includes the pump 46, the composite component 48, and the tube 54.

A specific description will be given. The tube 40 is connected to the base of the tank 50. The radiator 52 includes a tabular body expanding in the x direction and the y direction and the fin array protruding from the body. The tube 54 is provided between a discharge port of the radiator 52 and the pump 46. A tube 96 extending from the pump 46 is connected to the tube 38. According to the embodiment, a plurality of laid-pipe connections, namely, a plurality of joints in the connector is all centralized in a partial space 102 and a partial space 104. When viewed from above, the partial spaces 102 and 104 each are located apart from the space right above the ventilation area.

The blower fan 98 is provided adjacently to the intake portion 60. The blower fan 98 generates intake power and discharge power. The sucked air is guided to the discharge portion 62 through the inside of the duct member. The fin array is provided inside the duct member. The absorbing member 100 that is sheet-shaped is disposed on the upper face of the duct member such that the absorbing member 100 covers the entirety thereof. The absorbing member 100 is a spongy member as a porous member. Even if the coolant leaks at any of the joints, the leaking coolant is absorbed by the absorbing member 100. Note that, even in a case where the leaking coolant is not absorbed by the absorbing member 100 or even in a case where the absorbed coolant flows downward from the absorbing member 100, movement of the coolant from the non-ventilation area to the ventilation area is restricted by the inner step.

FIG. 5 illustrates the lower case 72. The lower case 72 is formed of the bottom wall 78 and a plurality of side walls 80A, 82A, 84A, and 86A. The inner face of the bottom wall 78 has the ventilation area 78A and the non-ventilation area 78B adjacent to the ventilation area 78A. The ventilation area 78A is provided with the intake portion 60. Specifically, the opening group 88 appears in the ventilation area 78A. In practice, it can be said that the ventilation area 78A is higher in height than the periphery. The inner step 90 is present between the ventilation area 78A and the non-ventilation area 78B.

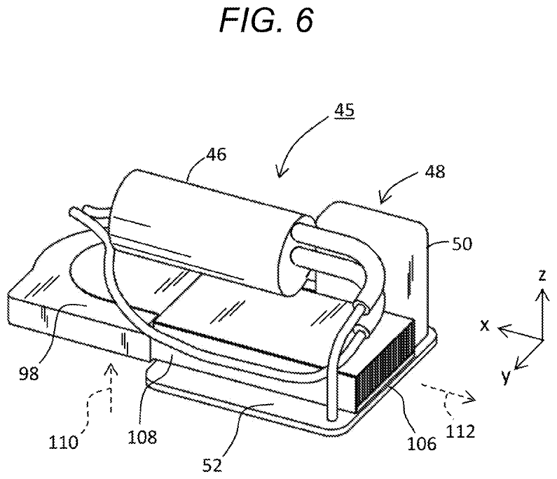

FIG. 6 illustrates an assembly to be provided in the housing. The assembly includes the coolant equipment 45. The coolant equipment 45 includes the pump 46 and the composite component 48. The pump 46 that is cylindrical is, for example, a polyphase diaphragm pump. The duct member 108 is provided in series with the blower fan 98. The fin array 106 is provided inside the duct member 108. Due to the effect of the blower fan 98, outer air is taken in the housing through the opening group for intake (refer to reference numeral 110). The air is guided to the discharge portion through the inside of the duct member 108, and then is discharged outside through the opening group for discharge (refer to reference numeral 112). During the process, the fin array 106 is cooled.

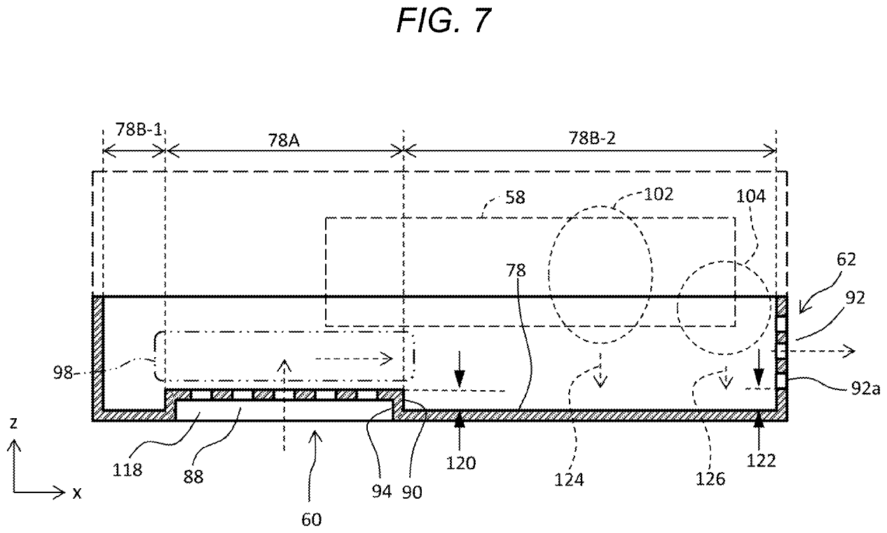

FIG. 7 illustrates a schematic section of the lower case. The intake portion 60 has the opening group 88 formed at the bottom wall 78. The inner face of the bottom wall 78 is broadly divided into the ventilation area 78A and the non-ventilation area 78B (specifically, 78B-1 and 78B-2). The ventilation area 78A is located higher by an offset 120 than the non-ventilation area 78B. The inner step 90 is present therebetween. Therefore, in a case where the connector is in the horizontal attitude, movement of the coolant from the non-ventilation area 78B to the ventilation area 78A is blocked by the inner step. Note that the offset 120 is, for example, several millimeters. For example, the offset 120 is in a range of 1 to 3 mm.

According to the embodiment, the volume of a recess corresponding to the non-ventilation area 78B is not less than the volume corresponding to the total amount of the coolant. This arrangement enables, even if all the coolant leaks into the non-ventilation area 78B, prevention or decrease of outward leakage of the coolant due to entry of the coolant into the ventilation area 78A. Note that a certain level of gap is present between the blower fan 98 and the ventilation area 78A.

A recess 118 recessed in the z direction is formed at the outer face of the bottom wall 78. The outer step 94 is present on the periphery of the recess 118. At grasping of the connector by a hand, if one or a plurality of fingers enters the recess 118, the outer step 94 acts as a catcher, resulting in improvement in grasp force. This arrangement enables, particularly at attachment of the connector and at detachment of the connector, improvement of the workability thereof. Note that, as illustrated, the outer step 94 is one size smaller than the inner step 90. Note that, in order to enhance the structure of the housing, one or a plurality of ribs may be provided to the bottom face of the bottom wall 78.

The discharge portion 62 has the opening group 92 formed at a specific side wall. The opening group 92 is provided higher by a certain distance than the inner face of the bottom wall 78. Specifically, in the illustrated exemplary configuration, an opening 92a lowest in location in the opening group 92 is formed at a location higher by an offset 122 than the inner face of the bottom wall 78. For example, the offset 122 is in size not less than the offset 120.

As illustrated in FIG. 7, the partial spaces 102 and 104 each including the plurality of joints are located apart from the space right above the ventilation area 78A. Specifically, the partial spaces 102 and 104 are set in the space right above the non-ventilation area 78B-2. Even if droplets 124 and 126 are generated due to leakage of the coolant in the partial spaces 102 and 104, basically, the droplets 124 and 126 drop to the non-ventilation area 78B-2. Even in consideration of movement of droplets through a member, the droplets are less likely to drop to the ventilation area 78A. According to the embodiment, because the absorbing member expanding horizontally is provided on the lower side of the partial spaces 102 and 104 and because intake is performed at the opening group 88, the coolant can be effectively prevented from leaking through the opening group 88, in cooperation with the effect of the weir structure above, namely, the effect of the inner step 90.

The substrate array 58 is located apart upward from the inner face of the bottom wall 78 in the housing. The substrate array 58 is formed of the plurality of substrates. Each substrate has a horizontal attitude. The substrate array 58 is provided such that the center of the substrate array 58 is higher in height than the center of the housing in the z direction. In this manner, the substrate array 58 is provided at a location biased upward in the housing, so that the substrate array 58 can be protected securely from the coolant leaking at ultrasonic diagnosis, namely, in the attached state. In the non-attached state, the substrate array 58 is easily protected from the coolant leaking. In consideration of such a case, desirably, the substrate array 58 is provided at a location biased upward such that the substrate array 58 is spaced apart from the upper face and the four side walls in addition to the bottom wall 78. Note that the inner case holding and housing the substrate array 58 is fixed to any of the side walls (e.g., the side wall on the device body side).

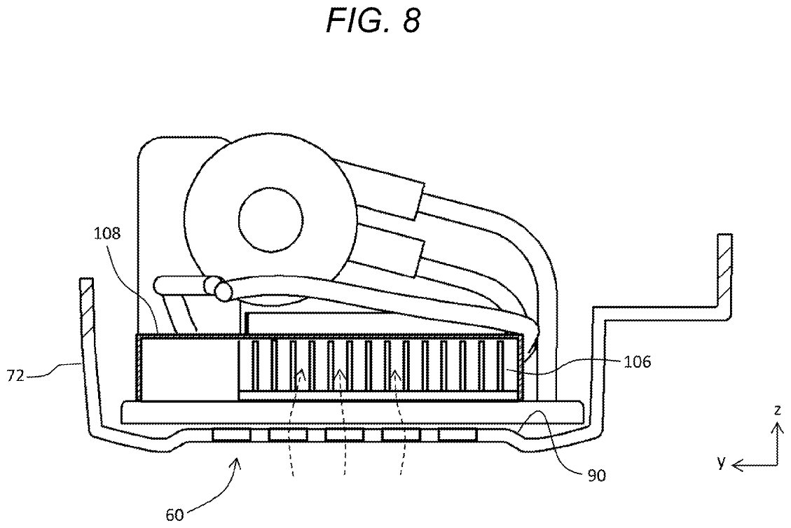

FIG. 8 specifically illustrates a yz section of the lower case 72. In practice, the inner step 90 provided on the periphery of the intake portion 60 has an oblique face instead of a vertical face. Such a configuration enables restriction of movement of the coolant. The non-ventilation area on the periphery of the inner step 90 has an oblique face. In the illustrated exemplary configuration, a wide V-shaped groove is formed by the oblique face of the inner step 90 and the peripheral oblique face. In a case where the coolant leaks, the coolant gathers in the hollow of the V-shaped groove. Note that FIG. 8 illustrates the fin array 106 provided in the duct member 108.

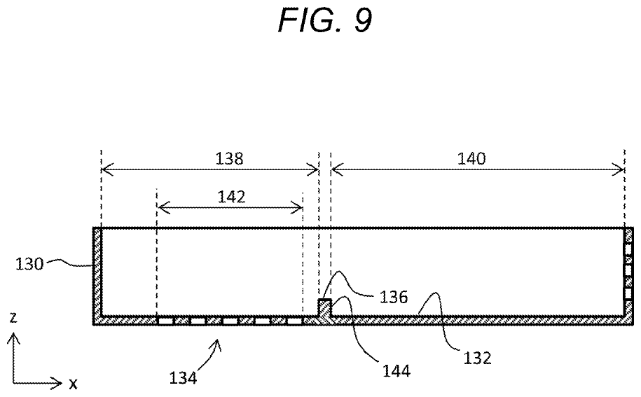

FIG. 9 illustrates a modification of the weir structure. A lower case 130 has a bottom wall 132. A bank 136 that functions as the weir structure, is provided at the bottom wall 132. The height of the bank 136 is, for example, several millimeters. The bank 136 is transversal in the y direction on the inner face of the bottom wall 132. A vertical face 144 on one side of the bank 136 achieves a function the same as that of the inner step above. The bank 136 is a partition or a wall. With the bank 136 as a boundary, the inner face of the bottom wall 132 is segmented into a first area 138 and a second area 140. The first area 138 includes an opening group 134, namely, a ventilation area 142. The second area 140 is a non-ventilation area. Such a weir structure enables restriction of movement of the coolant from the non-ventilation portion to the ventilation portion.

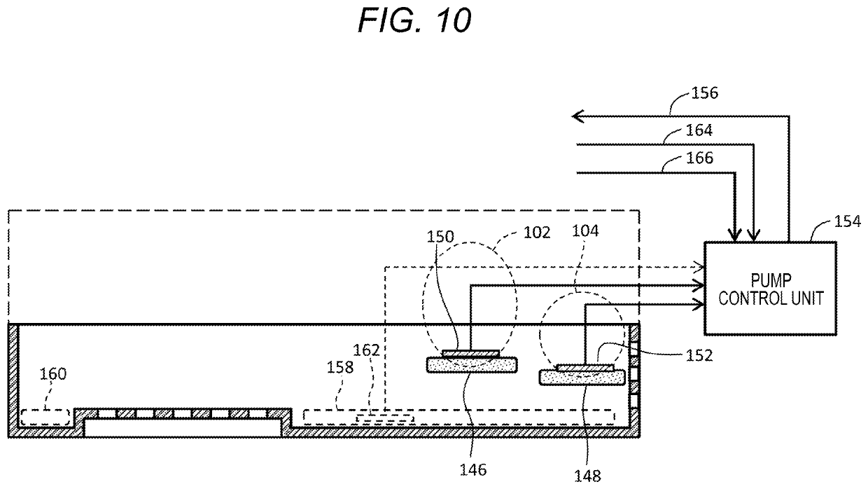

FIG. 10 schematically illustrates exemplary arrangement of absorbing members and a control method. As illustrated in FIG. 10, a plurality of absorbing members 146 and 148 may be provided, corresponding to the plurality of partial spaces 102 and 104, under the plurality of partial spaces 102 and 104. In that case, for each joint, a barrel-shaped absorbing member enveloping the joint may be provided. In the example illustrated in FIG. 10, sensors 150 and 152 are provided together with the plurality of absorbing members 146 and 148. The sensors 150 and 152 each detect leakage of the coolant, electrically.

A pump control unit 154 corresponds to one function of the system control unit illustrated in FIG. 1. The pump control unit 154 determines leakage of the coolant, on the basis of detected signals from the plurality of sensors 150 and 152, and performs control of stopping the pump, on the basis of the determination. A control signal 156 causes control of the operation of the pump. Control of reducing the number of revolutions of the pump, may be performed. In a case where leakage of the coolant is determined, desirably, an alarm is generated. In a case where one large sheet-shaped absorbing member is provided, one sensor may be provided at a place at which leakage is more likely to occur, or one or a plurality of sensors may be provided all over the absorbing member.

As indicated with broken lines in FIG. 10, absorbing members 158 and 160 may be provided all over the non-ventilation area of the bottom wall. In that case, the absorbing members 158 and 160 each may have a sensor embedded therein. As the sensor, a sensor that varies in resistance due to adhesion of the coolant may be used.

For example, the operation of the pump may be controlled by determination of deterioration in cooling capacity, based on an output signal 164 from a temperature sensor provided in the head. In a case where the operation of the pump is feedback-controlled by a pulse width modulation (PWM) system, deterioration in cooling capacity may be determined on the basis of a signal 166 indicating a duty cycle. Desirably, an absorbing member capable of absorbing the total amount of the coolant is provided in the housing. Note that the pump control unit 154 may be provided in the ultrasonic probe instead of being provided in the device body.

Next, the second embodiment will be described with FIGS. 11 to 14. For constituents equivalent to constituents in the first embodiment in the description of the second embodiment, the descriptions thereof will be omitted or the detailed descriptions thereof will be omitted.

FIG. 11 illustrates the connector 170. The connector 170 that is box-shaped or tabular, expands in the y direction and the z direction. In FIG. 11, the connector 170 is in a vertical attitude. With the vertical attitude retained, the connector 170 is attached to a device body. Alternatively, the connector 170 having been attached to the device body is in the vertical attitude.

The connector 170 includes a housing 172. The housing 172 has an upper wall 178, a lower wall (bottom wall) 180, and four side walls 182, 184, 186, and 188. The side wall 182 is provided with a terminal portion 190. In practice, the housing 172 is formed of a first case (right case) 174 and a second case (left case) 176.

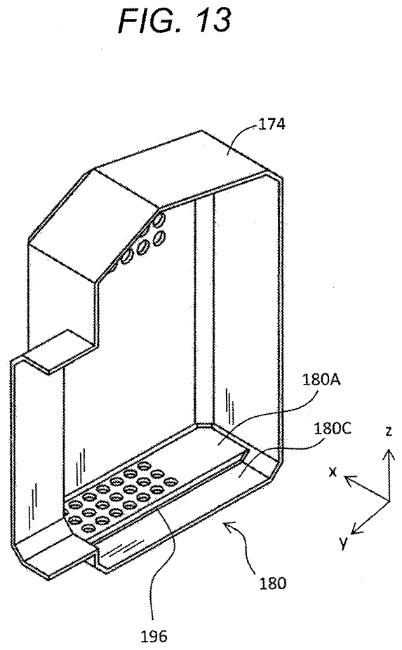

FIG. 12 illustrates the bottom face of the connector. The bottom wall 180 in the housing 172 has a ventilation portion 180A and a non-ventilation portion 180B. In the z direction, the ventilation portion 180A is located higher than the non-ventilation portion 180B. An outer step 183 is present therebetween. The ventilation portion 180A is provided with an opening group included in an intake portion 192. The side wall 188 is provided with an opening group included in a discharge portion 194.

FIG. 13 illustrates the first case 174. The bottom wall of the first case 174 has the ventilation portion 180A and a non-ventilation portion 180C. The non-ventilation portion 180C is part of the non-ventilation portion 180B illustrated in FIG. 12. In FIG. 13, an inner step 196 that functions as a weir structure or a weir means is present between the ventilation portion 180A and the non-ventilation portion 180C.

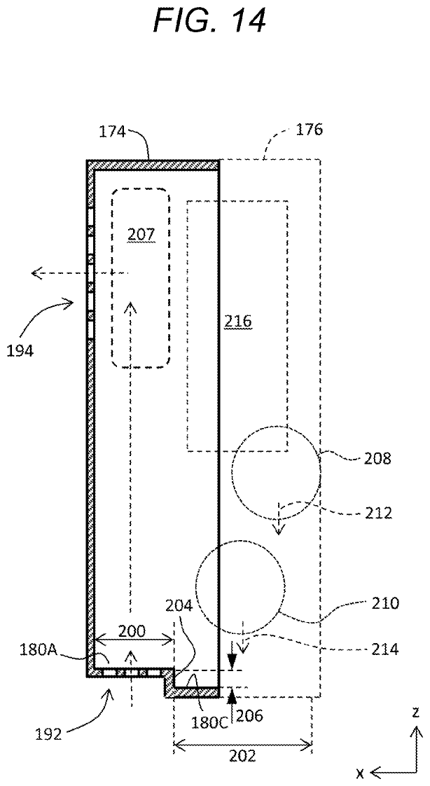

FIG. 14 illustrates an xz section of the first case 174. The housing is formed of the first case 174 and the second case 176. The intake portion 192 includes the ventilation portion 180A, and the inner face thereof forms a ventilation area 200. The inner face of the non-ventilation portion (refer to reference numeral 180B of FIG. 12) including the non-ventilation portion 180C, forms a non-ventilation area 202. The non-ventilation area 202 is adjacent to the ventilation area 200 through an inner step 204. The discharge portion 194 is formed at the upper portion of the first case 174. A blower fan 207 is provided in the first case 174. In the exemplary configuration illustrated in FIG. 14, the blower fan 207 is provided in proximity to the discharge portion 194, but the blower fan 207 may be provided in proximity to the intake portion 192. The intake portion 192 and the discharge portion 194 may be replaced in location.

Two partial spaces 208 and 210 each include an aggregation of a plurality of joints. Each joint in the housing belongs to either of the partial spaces 208 and 210. The two partial spaces 208 and 210 each are set at a location apart from the space right above the ventilation area 200. Specifically, the two partial spaces 208 and 210 are set in the space above the non-ventilation area 202. Therefore, even when the coolant leaks in the two partial spaces 208 and 210, resulting in generation of droplets 212 and 214, if the droplets drop without touching any other member, the droplets reach the non-ventilation area 202. That is dropping of the droplets onto the ventilation area 200 is avoided. In the second embodiment, desirably, an absorbing member that absorbs the coolant leaking is provided.

The ventilation area 200 is higher in height than the non-ventilation area 202, and thus the inner step 204 is present therebetween. An offset 206 therefor is illustrated. For example, the offset is several millimeters. Movement of the coolant from the non-ventilation area 202 to the ventilation area 200 is restricted by the inner step 204. Therefore, outward leakage of the coolant through the opening group at the intake portion 192 can be prevented or decreased. According to the second embodiment, the intake portion is provided at the bottom wall. In a case where a droplet enters each individual opening, upward levitation force acts on the droplet, so that leakage of the coolant can be effectively prevented or decreased.

In the housing in the vertical attitude, a substrate array 216 is provided at a location biased upward. Specifically, the substrate array 216 is provided such that, in the z direction, the height of the center of the substrate array 216 is not less than the height of the center of the housing. In accordance with the location of the terminal portion and other circumstances, the substrate array 216 can be provided at a lower portion in the housing. In that case, desirably, the substrate array 216 is provided apart by a certain distance from the bottom wall. Such a configuration facilitates protection of the substrate array from the coolant leaking.

According to the embodiment, the coolant-circulation ultrasonic probe enables, even if the coolant leaks in the connector, prevention of leakage of the coolant outside the connector or decrease of the possibility of the leakage. Therefore, safety can be maintained or improved.

What is claimed is:

1. An ultrasonic probe comprising:
a head configured to perform ultrasonic transmission and reception; and
a connector connected to the head through a cable, the connector being to be detachably attached to an ultrasonic diagnostic device body, the connector including coolant equipment in which a coolant for head cooling flows and a housing housing the coolant equipment, wherein
the housing has a bottom wall located at a lower portion in an attached state in which the connector is attached to the ultrasonic diagnostic device body,
the bottom wall has a ventilation portion having a plurality of openings and a non-ventilation portion adjacent to the ventilation portion, and
the bottom wall has a weir structure for restriction of movement of liquid from an inner face of the non-ventilation portion to an inner face of the ventilation portion in the attached state.

2. The ultrasonic probe according to claim 1, wherein
the inner face of the ventilation portion is located higher than the inner face of the non-ventilation portion in the attached state, and
the weir structure includes an inner step between the inner face of the ventilation portion and the inner face of the non-ventilation portion.

3. The ultrasonic probe according to claim 2, wherein
an outer face of the ventilation portion is located higher than an outer face of the non-ventilation portion in the attached state, and
an outer step is provided between the outer face of the ventilation portion and the outer face of the non-ventilation portion.

4. The ultrasonic probe according to claim 1, wherein
the coolant equipment includes a plurality of joints, and
the plurality of joints is located apart from space right above the inner face of the ventilation portion in the attached state.

5. The ultrasonic probe according to claim 1, wherein
a substrate array of a plurality of substrates is provided, and
the substrate array is deviated upward in location in the housing in the attached state.

6. The ultrasonic probe according to claim 1, wherein
an absorbing member that absorbs the coolant leaking is provided in the housing.

7. The ultrasonic probe according to claim 1, wherein
the ventilation portion is a ventilation portion for intake.

8. An ultrasonic diagnostic device comprising:
an ultrasonic diagnostic device body; and
an ultrasonic probe including: a head that performs ultrasonic transmission and reception; and a connector to be detachably attached to the ultrasonic diagnostic device body, wherein
the connector includes coolant equipment in which a coolant flows and a housing housing the coolant equipment,
the housing has a bottom wall located at a lower portion in an attached state in which the connector is attached to the ultrasonic diagnostic device body,
the bottom wall has a ventilation portion having a plurality of openings and a non-ventilation portion adjacent to the ventilation portion, and
the bottom wall has a weir structure for restriction of movement of liquid from an inner face of the non-ventilation portion to an inner face of the ventilation portion in the attached state.

9. The ultrasonic diagnostic device according to claim 8, wherein
the ultrasonic probe includes a detector that detects leakage of the coolant in the housing, and
the ultrasonic diagnostic device body or the ultrasonic probe includes a control unit that controls an operation of the coolant equipment, based on a detected signal output from the detector.
